(19) United States
(12) Patent Application Publication
(10) Pub. No.: US 2014/0248284 A1
(43) Pub. Date: Sep. 4, 2014

(54) METHODS FOR THE DETECTION AND THE TREATMENT OF CARDIAC REMODELING

(71) Applicants: INSERM (INSTITUT NATIONAL DE LA SANTE ET DE LA RECHERCHE MEDICALE), Paris (FR); UNIVERSITE DE LORRAINE, Nancy (FR); CENTRE HOSPITALIER ET UNIVERSITAIRE DE NANCY (CHU), Nancy Cedex (FR)

(72) Inventors: Faiez Zannad, Vandoeuvre Les Nancy (FR); Patrick Rossignol, Vandoeuvre Les Nancy (FR)

(21) Appl. No.: 14/352,735

(22) PCT Filed: Oct. 22, 2012
(86) PCT No.: PCT/EP2012/070882

§ 371 Date: Apr. 18, 2014

(30) Foreign Application Priority Data
Oct. 20, 2011 (EP) 11306354.9

(51) Int. Cl. C07K 16/40; G01N 33/573; A61K 45/06

(52) U.S. Cl. CPC C07K 16/40; A61K 45/06; G01N 33/573
USPC 424/158.1; 435/7.4

(57) ABSTRACT

The present invention relates to methods and kits for detecting cardiac remodeling in subjects without clinical signs of heart failure. The present invention also relates to methods and pharmaceutical compositions for the prevention and the treatment of cardiac remodeling.

### FIELD OF THE INVENTION

The present invention relates to methods and kits for detecting cardiac remodeling in subjects without clinical signs of heart failure.

The present invention also relates to methods and pharmaceutical compositions for the prevention and the treatment of cardiac remodeling.

### BACKGROUND OF THE INVENTION

Obesity has reached worldwide epidemic proportions. Moreover, the risk of developing chronic heart failure (CHF) is doubled in obese patients (1). Abdominal obesity (AO) is also a known cardiovascular risk factor (2, 3). The 2005 classification (4) of CHF edited by the ACC/AHA has insisted on the early stages (A and B) before any symptoms. However, the mechanisms underlying the transition to the symptomatic stages are still poorly known (1), whereas their understanding could open future perspectives for early detection of heart failure and preventive therapy in subjects without clinical signs of heart failure.

CHF is commonly secondary to cardiac remodeling (CR) except for acute cardiomyopathies. CR is usually defined as a dilatation of the left ventricle (LV) after adaptive hypertrophy and had been confirmed as a strong indicator of cardiovascular prognosis in the MESA (Multi-Ethnic Study of Atherosclerosis) study (5, 6), while LV mass (LVM) and LV hypertrophy (LVH) had been identified as an independent cardiovascular risk factor (7, 8). A cardiac magnetic resonance imaging (MRI) study had shown that subjects presenting with uncomplicated obesity exhibit a greater increase in LV wall thickness than in LV diameter (9).

In advanced stages of HF, there is an activation of the renin-angiotensin-aldosterone system (RAAS) (10). The degree of activation of the RAAS is all the more important when LV function is impaired in systolic HF (11). Aldosterone leads to LVH in untreated patients with essential hypertension probably by a myocardial fibrosis (12). The beneficial role of aldosterone receptor antagonists on morbidity and mortality in CHF patients (13, 14) and in post myocardial infarction (15) is now clearly established.

Aldosterone receptor antagonists have also demonstrated their impact on limiting extracellular matrix turnover (16-18), but also on diastolic dysfunction (DD) parameters in hypertensive heart disease with diastolic HF (19). Concurrently, there is evidence of hyperaldosteronism (22) in obese patients, possibly via activation of the systemic RAAS, but also of adipose-tissue pathways (23). This hyperaldosteronism regresses in obese patients after weight loss through dietary management, confirming the pejorative influence of adipose tissue (24).

There is no disclosure in the art of the use of an inhibitor of the renin angiotensin aldosterone system in methods for prevention and treatment of early cardiac remodeling, nor the use of an inhibitor of the renin angiotensin aldosterone system in methods for prevention and treatment of heart failure in subjects without clinical signs of heart failure.

However, there is a need to develop new drugs that will be suitable for prevention and treatment of cardiac remodeling and progression to heart failure in subjects afflicted with abdominal obesity.

### SUMMARY OF THE INVENTION

The present invention relates to a method for detecting cardiac remodeling in a subject without clinical signs of heart failure, comprising measuring renin level in a blood sample obtained from said subject.

### DETAILED DESCRIPTION OF THE INVENTION

The inventors have investigated the association between aldosterone, and renin plasma concentrations as well as their ratio and early alterations in cardiovascular structure and function in AO subjects age and gender matched with healthy volunteers. The inventors surprisingly found that renin is a determinant of LVM and cardiac remodeling in subjects with AO, at very early stages of HF (i.e without signs of HF), independently from blood pressure. It therefore appears imperative to seek better management of AO subjects, right from the earliest stages (stage A and B) of the disease (i.e. subjects without clinical signs of heart failure). It would therefore be particularly desirable to use inhibitors of the renin angiotensin aldosterone system for preventing the occurrence of LVH and/or of diastolic dysfunction, including in non-hypertensive patients.

### Prognostic and Diagnostic Methods According to the Invention

The present invention relates to a method for detecting cardiac remodeling in a subject without clinical signs of heart failure, comprising measuring renin level in a blood sample obtained from said subject.

As used herein, the phrase, “cardiac remodeling” refers to a compensatory physiologic response to an event or condition that compromises cardiac function. Triggers for cardiac remodeling include myocardial infarction, hypertension, wall stress, inflammation, pressure overload, and volume overload. Alterations in myocardial structure can occur as quickly as within a few hours of injury, and may progress over months and years. Thus, the phrase “cardiac remodeling” encompasses the global, cellular, and genetic changes that lead to alterations in the ventricular shape and function. While initially beneficial, these changes over time (months to years) can impair myocardial function to the point of chronic intractable heart failure. The hallmarks of cardiac remodeling are manifested as chamber dilation, increase in ventricular sphericity, and the development of interstitial and perivascular fibrosis. Increased sphericity is positively associated with mitral regurgitation. Ventricular dilation mainly results from cardiomyocyte hypertrophy and lengthening, and to a lesser extent from increases in the ventricular mass.

As used herein, the phrase “heart failure”, refers to any condition that can result from any structural or functional cardiac disorder that impairs the ability of the heart (e.g., the ventricle) to fill with or eject blood.

Typically a subject without clinical signs of heart failure is a subject classified at stage A of heart failure. The development of HF can be characterized by considering 4 stages of the disease. The first stage, Stage A, is a subject at high risk for HF but without structural heart disease or signs or symptoms of HF (for example, these are patients with hypertension, atherosclerotic disease, diabetes, obesity, metabolic syndrome or patients using cardiotoxins). The second stage, Stage B, is a subject having structural heart disease but without signs or symptoms of HF (for example, LV concentric remodeling as defined by an increased cardiac remodeling index represented by the ratio of LV mass/LV end-diastolic volume. The third stage, Stage C, is a subject having structural heart disease with prior or current symptoms and signs of HF (for example, these are patients who have known structural heart disease and exhibit shortness of breath and fatigue and have reduced exercise tolerance and display pulmonary rales and/or oedema). The fourth and final stage, Stage D, is refractory HF requiring specialized interventions (for example, patients who have marked symptoms at rest despite maximal medical therapy (namely, those who are recurrently hospitalized or cannot be safely discharged from the hospital without specialized interventions).

In a particular embodiment, the subject is an obese subject, and preferably a subject with abdominal obesity (>94 cm for male and >80 cm for female), and more preferably a subject with abdominal obesity without hypertension.

By “blood sample” is meant a volume of whole blood or fraction thereof, eg, serum, plasma, etc.

Measuring the renin level may be assessed by any of a wide variety of well-known methods for measuring proteins.

In a particular embodiment, the methods of the invention comprise contacting the blood sample with a binding partner capable of selectively interacting with renin present in the blood sample. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Alternatively, techniques described for the production of single chain antibodies (see e.g. U.S. Pat. No. 4,946,778) can be adapted to produce anti-renin, single chain antibodies. Antibodies useful in practicing the present invention also include anti-renin fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to renin. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

The afore mentioned assays generally involve the binding of the binding partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The level of biomarker protein may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

More particularly, an ELISA method can be used, wherein the wells of a microtiter plate are coated with a set of antibodies which recognize said biomarker protein. A blood sample containing or suspected of containing said biomarker protein is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In another embodiment, the renin level is assessed by analysing renin activity or plasma renin activity with an enzymatic method or a chemiluminescent assay as described for example in Marganti et al., 2010.

The method according to the invention may further comprise a step consisting of comparing the renin level in the blood sample with a reference value, wherein detecting differential in the renin level between the blood sample and the reference value is indicative of detecting cardiac remodeling in a subject without clinical signs of heart failure.

In one embodiment, the reference value may be index value or may be derived from one or more risk prediction algorithms or computed indices for cardiac remodeling. A reference value can be relative to a number or value derived from population studies, including without limitation, such subjects having similar body mass index, similar abdominal obesity, total cholesterol levels, LDL/HDL levels, systolic or diastolic blood pressure, subjects of the same or similar age range, subjects in the same or similar ethnic group. In one embodiment of the present invention, the reference value is derived from the level of renin in a control sample derived from one or more subjects who are substantially healthy (i.e. having no cardiac remodeling). In another embodiment, such subjects are monitored and/or periodically retested for a diagnostically relevant period of time (“longitudinal studies”) following such test to verify continued absence of cardiac remodeling. Such period of time may be one year, two years, two to five years, five years, five to ten years, ten years, or ten or more years from the initial testing date for determination of the reference value. Furthermore, retrospective measurement of renin levels in properly banked historical subject samples may be used in establishing these reference values, thus shortening the study time required, presuming the subjects have been appropriately followed during the intervening period through the intended horizon of the product claim. Typically, the levels of renin in a subject has cardiac remodeling is deemed to be higher than the reference value obtained from healthy subjects who have developed cardiac remodeling.

The method of the invention may also be particularly useful for monitoring the efficacy of a treatment for a cardiac remodeling.

The method of the invention is particularly suitable for the management and the appreciation of risk for heart failure. Accordingly, the present invention relates to a method for determining whether a subject is at risk of developing heart failure comprising a step consisting of detecting cardiac remodeling according to the method as above described.

### Therapeutic Methods and Uses

The present invention also relates to an inhibitor of the renin angiotensin aldosterone system for use in the prevention or treatment of cardiac remodeling in a subject without clinical signs of heart failure wherein said cardiac remodeling was previously detected in said subject by the method according to the invention.

In one embodiment, the present invention also relates to an inhibitor of the renin angiotensin aldosterone system for use in the prevention or treatment of cardiac remodeling in a subject without clinical signs of heart failure and not afflicted with arterial hypertension wherein said cardiac remodeling was previously detected in said subject by the method according to the invention.

In one embodiment, the present invention also relates to an inhibitor of the renin angiotensin aldosterone system for use in the prevention or treatment of heart failure in a subject not afflicted with arterial hypertension wherein said heart failure was previously detected in said subject by the method according to the invention.

The term “inhibitor of the renin angiotensin aldosterone system” has it general meaning in the art. Such inhibitors may include but are not limited to renin inhibitors; Angiotensin converting enzyme (ACE) inhibitors; Angiotensin-II-receptor blocker; Aldosterone synthase inhibitor; mineralocorticoid receptor antagonist; Dual angiotensin converting enzyme/neutral endopeptidase (ACE/NEP) inhibitor.

Examples of angiotensin converting enzyme (ACE) inhibitors include alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril.

Examples of Angiotensin-II-receptor blocker include valsartan, losartan, candesartan, telmisartan, eprosartan, olmesartan, irbesartan and tasosartan.

Examples of Aldosterone synthase inhibitor include non-steroidal aromatase inhibitors anastrozole, fadrozole (including the (+)-enantiomer thereof), as well as the steroidal aromatase inhibitor exemestane.

Examples of mineralocorticoid receptor antagonist include spironolactone and eplerenone.

Examples of Dual angiotensin converting enzyme/neutral endopeptidase (ACE/NEP) inhibitor include omapatrilate, fasidotril and fasidotrilate.

In a preferred embodiment, the inhibitor of the renin angiotensin aldosterone system according to the invention is a renin inhibitor.

The term “renin inhibitor” has it general meaning in the art, and refers to a natural or synthetic compound that inhibit the renin activity, or the renin expression, or the renin post-translational modification, or the renin secretory, or the renin enzyme function, or that have a biological effect opposite to that of the renin. In the context of the present invention, renin inhibitor of the invention is preferably selective for renin.

In one embodiment, the renin inhibitor is a low molecular weight inhibitor, e.g. a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Said renin inhibitors are well-known in the art as illustrated by Brown, (2008), Jensen et al., (2008), Barrios and Escobar, (2010), Wood et al., (1987).

In one embodiment of the invention, the renin inhibitor is selected from aliskiren and a salt thereof, especially a pharmaceutically acceptable salt thereof, most preferably a hemi-fumarate salt thereof and that have the chemical name 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide (WO 2008155338; WO 2007118023), ditekiren which has the chemical name [1S-[1R*,2R*,4R*(1R*,2R*)]]-1-[(1,1-dimethylethoxy) carbonyl]-L-proly I-L-phenylalanyl-N-[2-hydroxy-5-methyl-1-(2-methylpropyl)-4-[[[2-methyl-1-[[(2-pyridinylmethyl)amino]carbonyl]butyl]amino]carbonyl]hexyl]-N-alfa-methyl-L-histidinamide, terlakiren (chemical name: [R—(R*,S*)]-N-(4-morpholinylcarbonyl)-L-phenylalanyl-N-[1-(cyclohexylmethyl)-2-hydroxy-3-(1-methylethoxy)-3-oxopropyl]-5-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), remikiren, enalkiren, preferably, in each case, the hydrochloride salt thereof (EP 2275093), 1,3-Oxazolidine compounds (WO 2011056126), flavononol compounds (WO 2010077928), biaryl piperidine-based renin inhibitor compounds (WO 2010114978), 3,4-substituted piperidine derivatives (WO 2010066028) and pharmaceutically acceptable salts, solvates or derivatives of the above.

In another embodiment, the renin inhibitor may consist in an antibody or antibody fragment that can partially or completely block renin activity.

Non-limiting examples of antibody-based renin inhibitors include those described in Dzau et al., (1983), Dzau, (1985).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (1975). To prepare monoclonal antibodies useful in the invention, a mouse or other appropriate host animal is immunized at suitable intervals (e.g., twice-weekly, weekly, twice-monthly or monthly) with antigenic forms of renin. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in renin. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., (1999), the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′)2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Then after raising antibodies directed against renin as above described, the skilled man in the art can easily select those inhibiting renin.

In another embodiment the renin inhibitor is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against renin as above described, the skilled man in the art can easily select those inhibiting renin.

In another embodiment, the invention relates to an inhibitor of renin expression for use in the prevention or treatment of cardiac remodeling in a subject without clinical signs of heart failure having high level of renin.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., renin) modified by, for example, methylation, acetylation, phosphorylation, ubiquitination, SUMOylation, ADP-ribosylation, myristilation, and glycosylation.

An ‘inhibitor of expression” refers to a natural or synthetic compound that has a biological effect to inhibit the expression of a gene.

Inhibitors of renin expression for use in the present invention may be based on antisense oligonucleotide constructs. Anti-sense oligonucleotides, including anti-sense RNA molecules and anti-sense DNA molecules, would act to directly block the translation of renin mRNA by binding thereto and thus preventing protein translation or increasing mRNA degradation, thus decreasing the level of renin proteins, and thus activity, in a cell. For example, antisense oligonucleotides of at least about 15 bases and complementary to unique regions of the mRNA transcript sequence encoding renin can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of renin expression for use in the present invention. renin gene expression can be reduced by contacting the subject or cell with a small double stranded RNA (dsRNA), or a vector or construct causing the production of a small double stranded RNA, such that renin expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of renin expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of renin mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of renin expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing renin. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Chiffon, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In one embodiment, the present invention relates to a method of preventing or treating cardiac remodeling in a subject without clinical signs of heart failure in need thereof, wherein said cardiac remodeling was previously detected in said subject by the method according to the invention, comprising the step of administering to said subject a compound which is an inhibitor of the renin angiotensin aldosterone system.

In another embodiment, the present invention relates to a method of preventing or treating cardiac remodeling in a subject without clinical signs of heart failure and not afflicted with arterial hypertension in need thereof, wherein said cardiac remodeling was previously detected in said subject by the method according to the invention, comprising the step of administering to said subject a compound which is an inhibitor of the renin angiotensin aldosterone system.

In another embodiment, the present invention relates to a method of preventing or treating heart failure in a subject not afflicted with arterial hypertension in need thereof, wherein said heart failure was previously detected in said subject by the method according to the invention, comprising the step of administering to said subject a compound which is an inhibitor of the renin angiotensin aldosterone system.

In one embodiment said inhibitor of the renin angiotensin aldosterone system is a renin inhibitor.

### Pharmaceutical Compositions

Another object of the invention relates to a pharmaceutical composition comprising an inhibitor of the renin angiotensin aldosterone system and a pharmaceutically acceptable carrier for use in the prevention or treatment of cardiac remodeling in a subject without clinical signs of heart failure, wherein said cardiac remodeling was previously detected in said subject by the method according to the invention.

In another embodiment, the present invention also relates to a pharmaceutical composition comprising an inhibitor of the renin angiotensin aldosterone system and a pharmaceutically acceptable carrier for use in the prevention or treatment of cardiac remodeling in a subject without clinical signs of heart failure and not afflicted with arterial hypertension, wherein said cardiac remodeling was previously detected in said subject by the method according to the invention.

In another embodiment, the present invention also relates to a pharmaceutical composition comprising an inhibitor of the renin angiotensin aldosterone system and a pharmaceutically acceptable carrier for use in the prevention or treatment of heart failure in a subject not afflicted with arterial hypertension, wherein said heart failure was previously detected in said subject by the method according to the invention.

Typically, inhibitor of the renin angiotensin aldosterone system may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the renin angiotensin aldosterone system can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further agent which is used in the prevention or treatment of cardiac remodeling and heart failure. For example, the anti-cardiac remodeling and anti-heart failure may include but are not limited to Angiotensin-converting enzyme (ACE) inhibitors, Angiotensin II receptor blockers, Digoxin (Lanoxin), Beta blockers, Diuretics, Aldosterone antagonists, calcium channel blockers.

Pharmaceutical compositions of the invention may include any further agent which is used in the prevention or treatment of abdominal obesity.

In one embodiment, said additional active agents may be contained in the same composition or administrated separately.

In another embodiment, the pharmaceutical composition of the invention relates to combined preparation for simultaneous, separate or sequential use in the prevention or treatment of cardiac remodeling.

### Kits

A further object of the invention relates to kits for detecting cardiac remodeling in a subject without clinical signs of heart failure, comprising means for measuring renin level in a blood sample obtained from said subject.

In a particular embodiment, the kit of the invention further comprise means for comparing the renin level in the blood sample with a reference value, wherein detecting differential in the renin level between the blood sample and the reference value is indicative of detecting cardiac remodeling in a subject without clinical signs of heart failure.

A further object of the invention relates to kits comprising an inhibitor of the renin angiotensin aldosterone system for use in the prevention or treatment of cardiac remodeling in a subject without clinical signs of heart failure having high level of renin.

A further object of the invention relates to kits comprising a pharmaceutical composition according to the invention and a pharmaceutically acceptable carrier for use in the prevention or treatment of cardiac remodeling in a subject without signs of HF having high level of renin.

The invention will be further illustrated by the following examples. However, these examples should not be interpreted in any way as limiting the scope of the present invention.

### EXAMPLE

### Renin, a New Therapeutic Target in the Prevention of Cardiac Remodeling in Patients with Abdominal Obesity

Methods:

Subjects' Selection

Between 40 and 65 years AO subjects (waist circumference>94 cm for male, >80 cm for female (2)) and age-gender matched healthy volunteers (body mass index<25 kg/m²) without AO were consecutively recruited through press advertisement. Known diabetic subjects or participants, with known or suspected hypertension at the screening visit (i.e. BP>140/90 mm Hg), morbid obesity (body mass index>40 kg/m²), personal history of cardiovascular events or of endocrine disease, inflammatory or neoplasic diseases were excluded. Written informed consent was obtained. Ethic committee (CPP Est-III) gave his consent to this study. No clinical trials.gov number was assigned to this study since it started in 2005.

Cardiac Phenotyping

Transthoracic doppler echocardiography (TTE) (HDI 5000) Was performed and LV diastolic function was assessed with measurements of peak E wave, peak A wave, E/A ratio, deceleration time of E wave; doppler tissue imaging in the lateral part of mitral annulus: E′, A′ and E/E′ ratio. European society of echocardiography guidelines were used to classify DD (25). Diastolic dysfunction was diagnosed if E′ wave was less than 10 cm/s.

Cardiac MRI was performed on a 1.5-T magnet (Signa Excite, GE Medical Systems, Milwaukee, Wis., USA) equipped with an 8-element phased-array surface coil. A steady-state free precession pulse sequence was used to assess LV function in contiguous short axis planes, each slice being recorded during a ≦15-second breath-hold period (26, 27). Main acquisition parameters were as follows: 8 mm slice-thickness, 3.5-3.9 ms repetition time, 14 to 16 K-space lines per segment, 30 phases per cardiac cycle with view sharing, field-of-view (FOV) ranging from 32 to 38 cm with a phase FOV of 0.9, and a 224×224 matrix. LV end-diastolic volume, LV end-systolic volume, stroke volume, LV ejection fraction and LVM were determined on the contiguous SSFP short-axis slices, using dedicated software (MASS™, Medis, The Netherlands). LVM was determined at end-diastole, and papillary muscles and trabeculations were excluded for LVM and volume measurements (26, 27). CRI, indicating concentric LV remodeling, is represented by the ratio of LVM/LV end-diastolic volume (5, 6). Alfakih et al. defined LVH, assessed by MRI, as: women: ≧60 g/m² and men≧77 g/m² (28).

Determination of Fat-Free Mass (Dual-Energy X-Ray Absorptiometry (DEXA))

Body composition was estimated from the attenuation of X-rays pulsed synchronously between 40 and 100 keV using a LUNARS DPX-IQ system (LUNARS Corporation, Madison, Wis., USA) (29). Total fat mass, total lean mass and total bone mass were expressed in kg. Percentage of fat mass and fat free mass were evaluated using the proportions of total fat mass. Fat-free mass is an interesting criterion index in obese patients since it removes body fat. This index has never been used in studies of LVM by MRI but was in TTE (30, 31).

Vascular Phenotyping

Three consecutive BP measurements were performed first during the screening visit then before the echotracking and MRI (about one month apart), and the mean of the two last readings was recorded each time. Therefore, 4 measurements were taken into account in this analysis. All were performed after an extended rest period of at least 30 minutes. A diagnosis of hypertension was considered if the average of the 4 measurements was greater than 140/90 mmHg.

Carotid intima-media thickness and external pulse wave velocity were assessed as we previously reported (32).

Biological Phenotyping

Blood samples were taken off between 8 and 10 am after maintaining the supine position for 30 minutes: fasting glucose, oral glucose tolerance test (to exclude diabetic patients), and glycated haemoglobin serum creatinine [estimated glomerular filtration rate by the MDRD formula (33)], serum protein, lipoprotein A, ultra-sensitive C-reactive protein, lipid profile (triglycerides, total cholesterol, high density lipoprotein (HDL) and low density lipoprotein (LDL)), renin and aldosterone were measured. Samples were stored at −80° C. until assayed. We used dosage Coat-A-Count to determine plasma aldosterone concentrations (34) and the test Renin (35) Direct LINK (310 470, DiaSorin) to measure plasma renin concentration. They were performed by technicians blind for patients group.

Statistical Analysis

All tests were conducted using SAS software R9.1.3 (SAS Institute, Cary, N.C., USA). The bilateral significance level was set at 5%. The sample size allowed to detect with a 80% power a difference of 0.45 SD between groups.

Comparisons were carried out using the nonparametric Mann-Whitney.

Multivariate linear regressions were conducted on LVM (g, g/m² g/kg), CRI, pulse wave velocity, intima-media thickness, E′, E′<10 cm/s and E/E′. Significant covariates were selected using an interactive backward stepwise method. Intercorrelated variables (e.g. SBP, DBP and mean BP) were tested separately in the models. Each biomarker was tested individually in separate models. The conditions of validity of the models (linearity, normality of residuals, homoscedasticity, absence of interaction and collinearity, impact of outliers) were thoroughly checked for each model. The factors associated with DD were identified using logistic regression. When the assumption of linearity of the association between DD and continuous variable could not be accepted, the factor was dichotomized according to the median.

The results are presented as mean±standard deviation (m±SD), regression coefficient or odds ratio (confidence interval 95%). The means of renin, aldosterone plasma concentrations and aldosterone/renin ratios were computed after logarithmic-transformation and presented as mean (m−1 DS-m+1DS).

Results:

Study Population Features

192 subjects were recruited. Twenty-two (11 AO, 11 healthy volunteers) were excluded because of the finding of arterial hypertension during the initial visit (2 AO, 10 “healthy volunteers”), antihypertensive therapy (3 AO using beta-blockers and 1 healthy volunteers an α-blocker), thyroid hormone medication (6 AO), and one patient lost to follow-up between the two visits. Therefore, our final study population included 169 subjects: 116 subjects in the AO group (including 31 finally classified as untreated hypertensives after study completion and considered in the analysis), and 53 healthy volunteers.

The clinical, biological characteristics of both groups are presented in Table 1.

As expected from the inclusion criteria there was no significant difference regarding age and the gender ratio, whereas a significant increase in body mass index (p<0.0001), waist circumference (p<0.0001), and the waist/hip ratio (p<0.0001) were found in the AO population. On average, study participants did not exhibit BP in the hypertensive range, but SBP (p<0.0001) and DBP (p=0.0002) were significantly higher in the AO group, with also a higher heart rate (p<0.0001).

Aldosterone plasma level was about 70% higher in the AO group compared to healthy volunteers (p<0.0001), whereas renin plasma level was about 10% higher (p=0.12), with a 1.5-fold increase in the aldosterone/renin ratio (p=0.014).

The structural and functional cardiovascular data are summarized in Table 1. AO displayed a ventricular remodeling, as assessed by a significant increase in LVM (p=0.003), without reaching a LVH. Moreover, a CRI increase (p=0.004) secondary to the increase in LVM [no significant difference in LV end diastolic volume (p=0.90)] was observed in the AO group. The LV ejection fraction was preserved in both groups (p=0.98). E′ was significantly lower by about 30% in the AO group (p<0.0001). Forty-six percent of AO patients presented DD (grade I) compared to only 4% of controls. Two percent of AO group were already classified grade II, while 23% were in an intermediate group between groups I and II. Subjects with DD displayed a higher LVM (p=0.003) and a higher DBP (p<0.0001), while the latter remaining within the normal range (data not shown). Furthermore LV filling pressures (evaluated by E/E′ ratio) were significantly higher in obese patients (p=0.0003), but still in the normal range.

The two groups did not significantly differ in terms of vascular parameters [i.e. PWV (p=0.96), despite a trend toward increased intima-media thickness in the AO group (p=0.097)]. Only 5% and 1% of the total population respectively presented an elevation of the intima-media thickness (>0.90 mm) or of the PWV (>12 m/s) (37).

Association of Renin, Aldosterone and Aldosterone/Renin Ratio with Cardiovascular Remodeling

In multivariate analysis, plasma active renin concentration was positively associated (Table 2) to the LVM (p=0.036) [with similar trends when the LVM was adjusted for body surface area (p=0.073) or fat-free mass (p=0.083)] and LV concentric remodeling, as assessed by CRI (p=0.009). Aldosterone plasma concentration did not show any significant relationship with the different cardiovascular parameters [(LVM, CRI, PWV, E′, E/E′, despite a trend toward a negative association with E′ (p=0.086)]. The aldosterone/renin ratio was negatively associated to E′ (p=0.006) but not with DD (p=0.20). Hypertension (OR=3.78 (1.50-9.53), p=0.005) and AO (OR=3.95 (1.13-13.9), p=0.032) were positively associated with E/E′ ratio.

Finally there was a marginally significant positive relationship between aldosterone and intima-media-thickness (p=0.054).

Discussion:

The present study provides a number of novel findings. It first shows an association of the RAAS with early alterations in cardiac structure and function in asymptomatic AO subjects with normal BP levels on average. Indeed, AO subjects exhibited an increased LVM, without LVH, as well as a more pronounced concentric CR. Renin was positively associated with LVM and CRI. These findings were all observed after adjustment for conventional risk factors (metabolic syndrome components) and systemic inflammation, which were significantly higher in AO. Finally, we did not observe any modification in vascular structure and function in obese subjects.

RAAS, Cardiac Remodeling and LV Diastolic Dysfunction

Renin but not aldosterone (although there was a relative hyperaldosteronism without primary aldosteronism in AO participants), was positively associated with LVM and CR. To our knowledge, this is the first instance in which such a correlation is highlighted. In contrast, El-Gharbawy et al. (38) showed a negative correlation between LVM and plasma renin activity in obese hypertensive African American subjects off antihypertensive medications. One explanation for this discrepancy may be that our study population had not yet reached a hypertensive state on average, which is often associated with low renin in African American. Nevertheless, the association observed herein may reflect a direct effect of renin on heart muscle, since this effect was independent of either BP or myocardial fibrosis, as assessed by serum concentrations of collagen peptides (36). Corroborating this hypothesis, a pre-clinical study notably highlighted an increased expression of cardiac (pro)renin receptors in animals with CHF, along with an increase in angiotensin II (39). Moreover, the greater antihypertensive effect of direct renin inhibitors comparatively to that of hydrochlorothiazide in obese subjects with hypertension argues in favor of a significant deleterious involvement of renin in this particular population (40). Furthermore two clinical trials highlighted the potential benefit of addition of direct renin inhibitors on neurohumoral biomarkers in CHF (41, 42) but not after an acute myocardial infarction with systolic dysfunction to prevent CR (43).

Renin is a determinant of LVM and CR in subjects with AO, at very early stages of HF. It therefore appears necessary to seek better management of AO subjects, right from the earliest. At the present time, there is no treatment that has shown effectiveness in the treatment of diastolic HF. The use of direct renin inhibitors or more generally renin angiotensin aldosterone system inhibitors may represent a promising approach for preventing the occurrence of LVH and/or of DD, including in non-hypertensive on average AO patients.

TABLE 1
Study population features

| | Abdominal obesity group | | Control group | | |
|---|---|---|---|---|---|
| | n | Mean ± DS | n | Mean ± DS | p |
| Clinical characteristics | | | | | |
| Sex M/F | 116 | 50/50 | 53 | 45/55% | 0.57 |
| Age (years) | 116 | 55 ± 6 | 53 | 54 ± 6 | 0.18 |
| BMI (kg/m²) | 116 | 31.7 ± 3.4 | 53 | 22.4 ± 2.0 | <0.0001 |
| Body surface area (m²) | 116 | 2.07 ± 0.18 | 53 | 1.73 ± 0.14 | <0.0001 |
| Waist size (cm) | 116 | 103 ± 10 | 53 | 78 ± 8 | <0.0001 |
| Waist/hip ratio | 116 | 0.94 ± 0.10 | 53 | 0.82 ± 0.09 | <0.0001 |
| Mean SBP (mmHg) | 116 | 128 ± 16 | 53 | 116 ± 11 | <0.0001 |
| Mean DBP (mmHg) | 116 | 77 ± 11 | 53 | 71 ± 6 | 0.0002 |
| Mean MBP (mmHg) | 116 | 94 ± 12 | 53 | 86 ± 7 | <0.0001 |
| Mean HR (bpm) | 116 | 70 ± 10 | 53 | 62 ± 8 | <0.0001 |
| Biological characteristics | | | | | |
| Triglycerides (mmol/l) | 116 | 1.62 ± 1.16 | 53 | 0.88 ± 0.43 | <0.0001 |
| Total cholesterol (mmol/l) | 116 | 5.78 ± 0.99 | 53 | 5.44 ± 0.83 | 0.045 |
| HDL Cholesterol (mmol/l) | 116 | 1.42 ± 0.38 | 53 | 1.60 ± 0.36 | 0.002 |
| LDL Cholesterol (mmol/l) | 116 | 3.63 ± 0.88 | 53 | 3.45 ± 0.73 | <0.0001 |
| Fasting glucose (mmol/l) | 112 | 5.18 ± 0.95 | 49 | 4.43 ± 1.41 | 0.015 |
| Glycated haemoglobin (%) | 113 | 5.8 ± 0.5 | 50 | 5.6 ± 0.3 | 0.020 |
| CRP (mg/l) | 115 | 3.7 ± 5.5 | 52 | 1.2 ± 1.3 | <0.0001 |
| Protidemia (g/l) | 116 | 72 ± 4 | 52 | 69 ± 4 | <0.0001 |
| eGFR (MDRD, ml/min/1.73 m²) | 116 | 76 ± 10 | 53 | 77 ± 12 | 0.92 |
| Aldosterone (pg/ml) | 116 | 59 (33-106) | 53 | 34 (18-65) | <0.0001 |
| Active renin (μUI/ml) | 116 | 9.1 (3.9-21.5) | 53 | 8.2 (4.2-15.9) | 0.12 |
| Aldosterone/renin ratio | 116 | 6.4 (2.5-16.7) | 53 | 4.1 (1.6-10.6) | 0.014 |
| Cardiac phenotyping | | | | | |
| LVM (g)* | 93 | 97 ± 25 | 47 | 84 ± 21 | 0.003 |
| LVMi (g/m²)* | 93 | 47 ± 10 | 47 | 48 ± 10 | 0.21 |
| LVM FFM (DEXA) (g/kg)* | 93 | 1.79 ± 0.28 | 47 | 1.78 ± 0.28 | 0.86 |
| LVEF (%)* | 93 | 60 ± 6 | 47 | 59 ± 7 | 0.98 |
| LVEDV (ml)* | 93 | 142 ± 29 | 47 | 141 ± 25 | 0.90 |
| LVESV (ml)* | 93 | 58 ± 19 | 47 | 57 ± 15 | 0.93 |
| LVEV (ml)* | 93 | 84 ± 15 | 47 | 84 ± 17 | 0.83 |
| CRI = LVM/LVEDV (g/ml)* | 93 | 0.69 ± 0.16 | 47 | 0.60 ± 0.10 | 0.004 |
| E (cm/s) ^{†} | 116 | 66 ± 16 | 52 | 74 ± 15 | 0.005 |
| A (cm/s) ^{†} | 116 | 60 ± 15 | 52 | 53 ± 10 | 0.002 |
| E/A ^{†} | 116 | 1.13 ± 0.31 | 52 | 1.41 ± 0.31 | <0.0001 |
| E′ (cm/s) ^{†} | 112 | 10.4 ± 2.5 | 52 | 13.7 ± 2.5 | <0.0001 |
| A′ (cm/s) ^{†} | 113 | 11.0 ± 2.6 | 52 | 9.4 ± 2.6 | 0.0002 |
| E/E′ ^{†} | 113 | 6.6 ± 1.7 | 52 | 5.5 ± 1.5 | 0.0003 |
| Vascular phenotyping | | | | | |
| PWV (m/s) | 95 | 7.9 ± 1.7 | 48 | 8.0 ± 1.4 | 0.96 |
| IMT (mm) | 97 | 0.66 ± 0.15 | 48 | 0.61 ± 0.12 | 0.097 |

BMI: body mass index, CRP: C reactive protein, CRI = cardiac remodeling index, DBP: diastolic blood pressure, eGFR: estimated glomerular filtration rate, F: female, HDL: High density lipoprotein, HR: heart rate, bpm: beats per minute, IMT: intima-media thickness, LDL: Low density lipoprotein, LVEDV: LV end-diastolic volume, LVEF: left ventricular ejection fraction, LVESV: LV end-systolic volume, LVEV: LV ejection volume, LVM: left ventricular mass, M: male, LVM FFM: LVM indexed by fat free mass, LVMi: LVM indexed by BSA (Boyd), MBP: mean blood pressure, MDRD: Modification in Diet Renal Disease, PWV: pulse wave velocity, SBP: systolic blood pressure,
*evaluated by MRI
^{†} evaluated by TTE

TABLE 2
factors associated with left ventricular mass and
cardiac remodeling index in multivariate analysis.

| | Regression | | |
|---|---|---|---|
| | coefficients and | | |
| | confidence | | Variance |
| | interval 95% | p | explained* |
| LVM n = 140 | | | |
| Renin plasma | 382 (25-739) | 0.036 | 0.013 |
| concentrations | | | |
| (μUI/ml) | | | |
| Group (AO vs. HV) | −5.88 (−14.69-2.94) | 0.190 | 0.005 |
| Hypertension | 3.17 (−6.81-13.15) | 0.531 | 0.001 |
| (Yes vs. No) | | | |
| Sex (Female vs. Male) | −21.63 (−28.63-−14.64) | <0.0001 | 0.110 |
| BSA (m², Boyd) | 33.64 (13.31-53.97) | 0.001 | 0.031 |
| SBP (mmHg) | 0.40 (0.13-0.67) | 0.004 | 0.026 |
| Total variance explained by | | | 0.592 |
| the model (adjusted number of factors) | | | |
| CRI n = 135 | | | |
| Renin plasma | 3.70 (0.94-6.45) | 0.009 | 0.034 |
| concentrations | | | |
| (μUI/ml) | | | |
| Group (AO vs. HV) | 0.055 (0.009-0.102) | 0.021 | 0.026 |
| Hypertension | 0.006 (−0.062-0.074) | 0.863 | <0.001 |
| (Yes vs. No) | | | |
| Sex (Female vs. Male) | −0.092 (−0.19-−0.045) | 0.0002 | 0.072 |
| Hæmoglobin | 0.068 (0.004-0.132) | 0.038 | 0.021 |
| glycated (%) | | | |
| DBP (mmHg) | 0.004 (0.001-0.007) | 0.006 | 0.038 |

| | |
|---|---|
| Total variance explained by | 0.350 |
| the model (adjusted number of factors) | |

AO: abdominal obesity, BSA: body surface area, DBP: diastolic blood pressure, HV: Healthy volunteers, SBP: systolic blood pressure.
*Independently of other factors

### REFERENCES

Throughout this application, various references describe the state of the art to which this invention pertains. The disclosures of these references are hereby incorporated by reference into the present disclosure.
1. Kenchaiah S, Evans J C, Levy D, et al. Obesity and the risk of heart failure. N. Engl. J. Med 2002; 347(5):305-313.
2. Holt R I G. International Diabetes Federation re-defines the metabolic syndrome. Diabetes Obes Metab 2005; 7(5):618-620.
3. Jacobs E J, Newton C C, Wang Y, et al. Waist circumference and all-cause mortality in a large US cohort. Arch. Intern. Med. 2010; 170(15):1293-1301.
4. Hunt S A. ACC/AHA 2005 guideline update for the diagnosis and management of chronic heart failure in the adult: a report of the American College of Cardiology/American Heart Association Task Force on Practice Guidelines (Writing Committee to Update the 2001 Guidelines for the Evaluation and Management of Heart Failure). J. Am. Coll. Cardiol 2005; 46(6):e1-82.
5. Bluemke D A, Kronmal R A, Lima J A C, et al. The relationship of left ventricular mass and geometry to incident cardiovascular events: the MESA (Multi-Ethnic Study of Atherosclerosis) study. J. Am. Coll. Cardiol 2008; 52(25):2148-2155.
6. Cheng S, Fernandes V R S, Bluemke D A, McClelland R L, Kronmal R A, Lima J A C. Age-related left ventricular remodeling and associated risk for cardiovascular outcomes: the Multi-Ethnic Study of Atherosclerosis. Circ Cardiovasc Imaging 2009; 2(3):191-198.
7. Levy D, Garrison R J, Savage D D, Kannel W B, Castelli W P. Left ventricular mass and incidence of coronary heart disease in an elderly cohort. The Framingham Heart Study. Ann. Intern. Med 1989; 110(2):101-107. Available at: Consulté août 19, 2011.
8. Kannel W B, Gordon T, Castelli W P, Margolis J R. Electrocardiographic left ventricular hypertrophy and risk of coronary heart disease. The Framingham study. Ann. Intern. Med 1970; 72(6):813-822.
9. Turkbey E B, McClelland R L, Kronmal R A, et al. The impact of obesity on the left ventricle: the Multi-Ethnic Study of Atherosclerosis (MESA). JACC Cardiovasc Imaging 2010; 3(3):266-274.
10. Ma T K W, Kam K K H, Yan B P, Lam Y-Y. Renin-angiotensin-aldosterone system blockade for cardiovascular diseases: current status. Br. J. Pharmacol 2010; 160(6):1273-1292.
11. Benedict C R, Johnstone D E, Weiner D H, et al. Relation of neurohumoral activation to clinical variables and degree of ventricular dysfunction: a report from the Registry of Studies of Left Ventricular Dysfunction. SOLVD Investigators. J. Am. Coll. Cardiol 1994; 23(6):1410-1420.
12. Nakahara T, Takata Y, Hirayama Y, et al. Left ventricular hypertrophy and geometry in untreated essential hypertension is associated with blood levels of aldosterone and procollagen type III amino-terminal peptide. Circ. J 2007; 71(5):716-721.
13. Pitt B, Zannad F, Remme W J, et al. The effect of spironolactone on morbidity and mortality in patients with severe heart failure. Randomized Aldactone Evaluation Study Investigators. N. Engl. J. Med 1999; 341(10):709-717.
14. Zannad F, McMurray J J V, Krum H, et al. Eplerenone in patients with systolic heart failure and mild symptoms. N. Engl. J. Med 2011; 364(1):11-21. Available at: Consulté avril 10, 2011.
15. Pitt B, Remme W, Zannad F, et al. Eplerenone, a selective aldosterone blocker, in patients with left ventricular dysfunction after myocardial infarction. N. Engl. J. Med 2003; 348(14): 1309-1321.
16. Zannad F, Alla F, Dousset B, Perez A, Pitt B. Limitation of excessive extracellular matrix turnover may contribute to survival benefit of spironolactone therapy in patients with congestive heart failure: insights from the randomized aldactone evaluation study (RALES). Rales Investigators. Circulation 2000; 102(22):2700-2706.
18. MacFadyen R J, Barr C S, Struthers A D. Aldosterone blockade reduces vascular collagen turnover, improves heart rate variability and reduces early morning rise in heart rate in heart failure patients. Cardiovasc. Res 1997; 35(1):30-34.
19. Mottram P M, Haluska B, Leano R, Cowley D, Stowasser M, Marwick T H. Effect of aldosterone antagonism on myocardial dysfunction in hypertensive patients with diastolic heart failure. Circulation 2004; 110(5):558-565.
22. Krug A W, Ehrhart-Bornstein M. Adrenocortical dysfunction in obesity and the metabolic syndrome. Horm. Metab. Res 2008; 40(8):515-517.
23. Schinner S, Willenberg H S, Krause D, et al. Adipocyte-derived products induce the transcription of the StAR promoter and stimulate aldosterone and cortisol secretion from adrenocortical cells through the Wnt-signaling pathway. Int J Obes (Lond) 2007; 31(5):864-870.
24. Engeli S, Bohnke J, Gorzelniak K, et al. Weight loss and the renin-angiotensin-aldosterone system. Hypertension 2005; 45(3):356-362.
26. Codreanu A, Djaballah W, Angioi M, et al. Detection of myocarditis by contrast-enhanced MRI in patients presenting with acute coronary syndrome but no coronary stenosis. J Magn Reson Imaging 2007; 25(5):957-964.
27. Mandry D, Lapicque F, Odille F, et al. Multicompartmental analysis of late contrast enhancement in areas of myocardial infarction supplied by chronically occluded coronary arteries. J Magn Reson Imaging 2009; 29(1):78-85.
28. Alfakih K, Plein S, Bloomer T, Jones T, Ridgway J, Sivananthan M. Comparison of right ventricular volume measurements between axial and short axis orientation using steady-state free precession magnetic resonance imaging. J Magn Reson Imaging 2003; 18(1):25-32.
29. He M, Li E T, Kung A W. Dual-energy X-ray absorptiometry for body composition estimation in Chinese women. Eur J Clin Nutr 1999; 53(12):933-937.
30. Quilliot D, Alla F, Böhme P, et al. Myocardial collagen turnover in normotensive obese patients: relation to insulin resistance. Int J Obes (Lond) 2005; 29(11):1321-1328.
31. Bella J N, Devereux R B, Roman M J, et al. Relations of left ventricular mass to fat-free and adipose body mass: the strong heart study. The Strong Heart Study Investigators. Circulation 1998; 98(23):2538-2544.
32. Kearney-Schwartz A, Rossignol P, Bracard S, et al. Vascular structure and function is correlated to cognitive performance and white matter hyperintensities in older hypertensive patients with subjective memory complaints. Stroke 2009; 40(4):1229-1236.
33. Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann. Intern. Med 1999; 130(6):461-470.
34. Kubasik N P, Warren K, Sine H E. Evaluation of a new commercial radioassay kit for aldosterone using an iodinated tracer. Clin. Biochem 1979; 12(2):59-61.
35. Morganti A. A comparative study on inter and intralaboratory reproducibility of renin measurement with a conventional enzymatic method and a new chemiluminescent assay of immunoreactive renin. J. Hypertens 2010; 28(6):1307-1312.
36. Zannad F, Rossignol P, Iraqi W. Extracellular matrix fibrotic markers in heart failure. Heart Fail Rev 2010; 15(4):319-329.
37. Mancia G, De Backer G, Dominiczak A, et al. 2007 Guidelines for the management of arterial hypertension: The Task Force for the Management of Arterial Hypertension of the European Society of Hypertension (ESH) and of the European Society of Cardiology (ESC). Eur. Heart J 2007; 28(12):1462-1536.
38. El-Gharbawy A H, Nadig V S, Kotchen J M, et al. Arterial pressure, left ventricular mass, and aldosterone in essential hypertension. Hypertension 2001; 37(3):845-850.
39. Hirose T, Mori N, Totsune K, et al. Gene expression of (pro)renin receptor is upregulated in hearts and kidneys of rats with congestive heart failure. Peptides 2009; 30(12):2316-2322.
40. Schmieder R E, Philipp T, Guerediaga J, Gorostidi M, Bush C, Keefe D L. Aliskiren-based therapy lowers blood pressure more effectively than hydrochlorothiazide-based therapy in obese patients with hypertension: sub-analysis of a 52-week, randomized, double-blind trial. J. Hypertens 2009; 27(7):1493-1501.
41. Pitt B, Latini R, Maggioni A P, et al. Neurohumoral effects of aliskiren in patients with symptomatic heart failure receiving a mineralocorticoid receptor antagonist: the Aliskiren Observation of Heart Failure Treatment study. Eur. J. Heart Fail 2011; 13(7):755-764.
42. McMurray J J V, Pitt B, Latini R, et al. Effects of the oral direct renin inhibitor aliskiren in patients with symptomatic heart failure. Circ Heart Fail 2008; 1(1):17-24.
43. Solomon S D, Hee Shin S, Shah A, et al. Effect of the direct renin inhibitor aliskiren on left ventricular remodelling following myocardial infarction with systolic dysfunction. Eur Heart J 2011.
Brown M J. Aliskiren. Circulation 2008; 118: 773-784.
Jensen C, Herold P, Brunner H R. Aliskiren: the first renin inhibitor for clinical treatment. Nat Rev Drug Discov 2008; 7: 399-410.
Barrios V, Escobar C. Aliskiren in the management of hypertension. Am J Cardiovasc Drugs. 2010; 10(6):349-58.
Wood J M, Stanton J L, Hofbauer K G. Inhibitors of renin as potential therapeutic agents. J Enzyme Inhib. 1987; 1(3):169-85.
Dzau V J, Devine D, Mudgett-Hunter M, Kopelman R I, Barger A C, Haber E. Antibodies as specific renin inhibitors: studies with polyclonal and monoclonal antibodies and Fab fragments. Clin Exp Hypertens A. 1983; 5(7-8):1207-20.
Dzau V J. In vivo inhibition of renin by antirenin antibodies: potential experimental and clinical applications. J Cardiovasc Pharmacol. 1985; 7 Suppl 4:S53-7.

1. A method for detecting cardiac remodeling in a subject without clinical signs of heart failure, comprising
measuring a renin level in a blood sample obtained from said subject.

2. The method according to claim 1 further comprising a step of comparing the renin level in the blood sample with a reference value, wherein detecting a differential between the renin level in the blood sample and the reference value is indicative of detecting cardiac remodeling in a subject without clinical signs of heart failure.

3. A method of preventing or treating cardiac remodeling in a subject without clinical signs of heart failure in need thereof, comprising the step of administering to said subject a compound which is an inhibitor of the renin angiotensin aldosterone system.

4. The method according to claim 3 wherein said inhibitor of the renin angiotensin aldosterone system is a renin inhibitor.

5. The method of claim 1, wherein said step of measuring is performed by
contacting the blood sample with an antibody capable of selectively interacting with renin present in the blood sample, thereby forming a renin-antibody complex; and
detecting the renin-antibody complex.

6. The method of claim 3, further comprising a step of
prior to said step of administering, detecting cardiac remodeling in the subject by measuring a renin level in a blood sample obtained from the subject.
